(19) United States
(12) Patent Application Publication
(10) Pub. No.: US 2021/0205013 A1
(43) Pub. Date: Jul. 8, 2021

(54) ENDOSCOPIC ULTRASOUND-GUIDED CELIAC PLEXUS ABLATION AND SENSING DEVICE

(71) Applicants: Boston Scientific Scimed, Inc., Maple Grove, MN (US); REGENTS OF THE UNIVERSITY OF MINNESOTA, Minneapolis, MN (US)

(72) Inventors: Audrey Francis Meyer, Rogers, MN (US); Heidi Ann Schwanz, Maple Grove, MN (US); Amy Lynn Hoelscher, Maplewood, MN (US); Lyndsey Ellen Calvin, St. Paul, MN (US); Kieran Gary Leong, Baltimore, MD (US); Yasheen Brijlal, Durban (ZA)

(21) Appl. No.: 17/137,705

(22) Filed: Dec. 30, 2020

Related U.S. Application Data
(60) Provisional application No. 62/956,807, filed on Jan. 3, 2020.
(60) Provisional application No. 62/961,000, filed on Jan. 14, 2020.

(51) Int. Cl. A61B 18/14

(52) U.S. Cl. CPC A61B 18/1492; A61B 2018/00994

(57) ABSTRACT

Embodiments herein relate to systems and methods for endoscopic ultrasound-guided celiac plexus ablation and sensing. In an embodiment, a system for endoscopic ultrasound-guided ablation and sensing is included having an ultrasound endoscope that can include an ultrasound transducer and a radiofrequency catheter configured to be disposed within the ultrasound endoscope. The radiofrequency catheter can include a first electrode pair disposed on the radiofrequency catheter, where the first electrode pair configured to be placed at or near at a site of a celiac plexus. The system is configured to sense a baseline electrical property to verify placement of the first electrode pair at the celiac plexus; deliver a radiofrequency energy to the celiac plexus through the first electrode pair; and sense a post-treatment electrical property of the celiac plexus to verify neurolysis of the celiac plexus. Other embodiments are also included herein.

This application claims the benefit of U.S. Provisional Application No. 62/956,807, filed Jan. 3, 2020, and U.S. Provisional Application No. 62/961,000, filed Jan. 14, 2020, the contents of which are herein incorporated by reference in their entirety.

### FIELD

Embodiments herein relate to systems and methods for endoscopic ultrasound-guided celiac plexus ablation and sensing. More specifically, embodiments herein relate to systems and methods for endoscopic ultrasound-guided celiac plexus ablation and sensing through the stomach or intestinal wall using combination ablation and sensing electrodes.

### BACKGROUND

A variety of foregut cancers and upper abdominal pain disorders can cause extreme pain and discomfort for a patient afflicted with these conditions. Various cancers that can cause intractable upper abdominal pain can include, stomach cancer, pancreatic cancer, gallbladder cancer, liver cancer, large or small intestine cancers, bile duct cancer, and the like. Various gastrointestinal disorders that can similarly lead to intractable abdominal pain can include, acute or chronic pancreatitis, Crohn's disease, inflammatory bowel disease, ulcerative colitis, and the like.

Traditional ablation methods can include chemical ablation of one or more nerve tissues associated with the celiac plexus, however, these have been found to be highly variable with respect to effectiveness and duration of symptom relief. Other methods of pain management include use of opioids, which can cause uncomfortable or serious side effects for the patient, including lethargy, constipation, nausea, vomiting, rash, paralysis, or addiction. Thus, a need exists for more effective pain management of such disorders with fewer deleterious side effects caused by the treatment modality.

### SUMMARY

Embodiments herein relate to systems and methods for endoscopic ultrasound-guided celiac plexus ablation and sensing. In a first aspect, a system for endoscopic ultrasound-guided ablation and sensing is included. The system can include an ultrasound endoscope having an ultrasound transducer and a radiofrequency catheter configured to be disposed within the ultrasound endoscope. The radiofrequency catheter can include a first electrode pair disposed on the radiofrequency catheter, where the first electrode pair can be configured to be placed at or near at a site of a celiac plexus. The system can be configured to sense a baseline electrical property to verify placement of the first electrode pair at the celiac plexus, deliver a radiofrequency energy to the celiac plexus through the first electrode pair, and sense a post-treatment electrical property of the celiac plexus to verify neurolysis of the celiac plexus.

In a second aspect, in addition to one or more of the preceding or following aspects, or in the alternative to some aspects, the system further can include a system controller in electrical communication with the ultrasound endoscope and the radiofrequency catheter.

In a third aspect, in addition to one or more of the preceding or following aspects, or in the alternative to some aspects, wherein the baseline electrical property is at least one of an action potential and an impedance.

In a fourth aspect, in addition to one or more of the preceding or following aspects, or in the alternative to some aspects, wherein the post-treatment electrical property is at least one of an action potential and an impedance.

In a fifth aspect, in addition to one or more of the preceding or following aspects, or in the alternative to some aspects, wherein delivering a radiofrequency energy to the celiac plexus includes the first electrode pair being configured to deliver an alternating electric current pulse or a voltage pulse having a frequency of from 250 kilohertz (kHz) to 3 megahertz (MHz) to the site of the celiac plexus.

In a sixth aspect, in addition to one or more of the preceding or following aspects, or in the alternative to some aspects, wherein delivering a radiofrequency energy to the celiac plexus includes the first electrode pair being configured to deliver an alternating electric current pulse or a voltage pulse having a frequency of from 250 kHz to 750 kHz to the site of the celiac plexus.

In a seventh aspect, in addition to one or more of the preceding or following aspects, or in the alternative to some aspects, wherein the radiofrequency catheter includes a needle catheter at a distal tip of the radiofrequency catheter.

In an eighth aspect, in addition to one or more of the preceding or following aspects, or in the alternative to some aspects, wherein the radiofrequency catheter includes more than one needle catheters at a distal tip of the radiofrequency catheter.

In a ninth aspect, in addition to one or more of the preceding or following aspects, or in the alternative to some aspects, wherein the radiofrequency catheter further includes at least one of a second electrode pair, a third electrode pair, or a fourth electrode pair.

In a tenth aspect, in addition to one or more of the preceding or following aspects, or in the alternative to some aspects, wherein the first electrode pair includes at least one of circular band pairs, interdigitated ring pairs, concentric ring-disk pairs, or parallel pairs.

In an eleventh aspect, in addition to one or more of the preceding or following aspects, or in the alternative to some aspects, wherein the first electrode pair is made of one or more materials can include silver, gold, platinum, iridium, glassy carbon, or iridium oxide-coated stainless steel, or derivatives or alloys thereof.

In a twelfth aspect, in addition to one or more of the preceding or following aspects, or in the alternative to some aspects, the radiofrequency catheter further can include at least one reference electrode.

In a thirteenth aspect, a method for endoscopic ultrasound-guided ablation and sensing in a subject is included. The method can include inserting an ultrasound endoscope and a radiofrequency catheter into a stomach or a small intestine of the subject at or near a site of a celiac plexus. The ultrasound endoscope of the methods herein can include an ultrasound transducer. The radiofrequency catheter is configured to be disposed within the ultrasound endoscope, where the radiofrequency catheter can include a first electrode pair disposed on the radiofrequency catheter, and the first electrode pair is configured to be placed at or near the site of the celiac plexus. The method includes advancing the radiofrequency catheter through a stomach wall or an intestinal wall to the site of the celiac plexus, sensing a baseline electrical property to verify placement of the first electrode pair at the celiac plexus, delivering a radiofrequency energy to the celiac plexus through the first electrode pair, and sensing a post-treatment electrical property of the celiac plexus to verify neurolysis of the celiac plexus.

In a fourteenth aspect, in addition to one or more of the preceding or following aspects, or in the alternative to some aspects, the method further can include delivering the radiofrequency energy to the celiac plexus until an electrical property of the celiac plexus reaches a predetermined value indicative of neurolysis.

In a fifteenth aspect, in addition to one or more of the preceding or following aspects, or in the alternative to some aspects, wherein the baseline electrical property is at least one of an action potential and an impedance.

In a sixteenth aspect, in addition to one or more of the preceding or following aspects, or in the alternative to some aspects, wherein the post-treatment electrical property is at least one of an action potential and an impedance.

In a seventeenth aspect, in addition to one or more of the preceding or following aspects, or in the alternative to some aspects, further can include delivering the radiofrequency energy to the celiac plexus until a post-treatment action potential signal of the celiac plexus reaches a value of less than 10% of a baseline action potential of the celiac plexus.

In an eighteenth aspect, in addition to one or more of the preceding or following aspects, or in the alternative to some aspects, wherein delivering a radiofrequency energy to the celiac plexus includes delivering alternating electric current pulse or a voltage pulse having a frequency of from 250 kilohertz (kHz) to 3 megahertz (MHz) to the site of the celiac plexus.

In a nineteenth aspect, in addition to one or more of the preceding or following aspects, or in the alternative to some aspects, the method further can include sensing an impedance of the celiac plexus by delivering an alternating current of from 1 to 10 (milliamp) mAmp through a range of frequencies of from 1 hertz (Hz) to 1000 Hz to the site of the celiac plexus.

In a twentieth aspect, in addition to one or more of the preceding or following aspects, or in the alternative to some aspects, the method further can include sensing an impedance of the celiac plexus using an electrochemical impedance spectroscopy.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE FIGURES

Aspects may be more completely understood in connection with the following figures (FIGS.), in which:

FIG. 1 is a schematic anterolateral view of a celiac plexus treatment site in accordance with various embodiments herein.

FIG. 2 is a schematic view of the components of a system in accordance with various embodiments herein.

FIG. 3 is a schematic view of the ultrasound endoscope and radiofrequency catheter in accordance with various embodiments herein.

FIG. 4 is a schematic view of a distal tip of a radiofrequency catheter in accordance with various embodiments herein.

FIG. 5 a schematic view of an alternative embodiment of a distal tip of a radiofrequency catheter in accordance with various embodiments herein.

FIG. 6 is a schematic view of an alternative embodiment of a distal tip of a radiofrequency catheter in accordance with various embodiments herein.

FIG. 7 is a schematic view of an alternative embodiment of a distal tip of a radiofrequency catheter in accordance with various embodiments herein.

FIG. 8 a schematic view of an alternative embodiment of a distal tip of a radiofrequency catheter in accordance with various embodiments herein.

FIG. 9 is a schematic view of the components of a system at or near a location of a treatment site in accordance with various embodiments herein.

FIG. 10 a flow diagram of a method in accordance with various embodiments herein.

FIG. 11 is a schematic view of elements of a system for endoscopic ultrasound-guided ablation and sensing in accordance with some embodiments herein.

FIG. 12 is an exemplary chronoamperometry scan of the celiac plexus during a treatment procedure in accordance with some embodiments herein.

FIG. 13 is an exemplary chronoamperometry scan of the celiac plexus during a treatment procedure in accordance with some embodiments herein.

FIG. 14 is an exemplary chronoamperometry scan of the celiac plexus during a treatment procedure in accordance with some embodiments herein.

FIG. 15 is an exemplary chronoamperometry scan of the celiac plexus during a treatment procedure in accordance with some embodiments herein.

FIG. 16 an exemplary chronoamperometry scan of the celiac plexus during a treatment procedure in accordance with some embodiments herein.

FIG. 17 an exemplary chronoamperometry scan of control tissue during a treatment procedure in accordance with some embodiments herein.

FIG. 18 is an exemplary chronoamperometry scan of control tissue during a treatment procedure in accordance with some embodiments herein.

FIG. 19 is exemplary chronoamperometry scan of control tissue during a treatment procedure in accordance with some embodiments herein.

FIG. 20 is exemplary chronoamperometry scan of control tissue during a treatment procedure in accordance with some embodiments herein.

FIG. 21 an exemplary open circuit potential scan of the celiac plexus during a treatment procedure in accordance with some embodiments herein.

FIG. 22 an exemplary open circuit potential scan of control tissue during a treatment procedure in accordance with some embodiments herein.

FIG. 23 is an exemplary electrochemical impedance scan of control tissue during a treatment procedure in accordance with some embodiments herein.

FIG. 24 is exemplary electrochemical impedance scan of control tissue during a treatment procedure in accordance with some embodiments herein.

FIG. 25 is exemplary electrochemical impedance scan of control tissue during a treatment procedure in accordance with some embodiments herein.

FIG. 26 is exemplary electrochemical impedance scan of control tissue during a treatment procedure in accordance with some embodiments herein.

While embodiments are susceptible to various modifications and alternative forms, specifics thereof have been shown by way of example and drawings, and will be described in detail. It should be understood, however, that the scope herein is not limited to the particular aspects described. On the contrary, the intention is to cover modifications, equivalents, and alternatives falling within the spirit and scope herein.

### DETAILED DESCRIPTION

As discussed above, various cancers and disorders of the foregut can cause extreme pain and discomfort to affected individuals. One avenue for treatment is neuromodulation of the nerve tissue at the site of the celiac plexus using chemical agents such as ethanol or phenol. Neuromodulation of a nerve tissue can lead to an alteration of nerve conductance associated with pain or abnormal neural activity, and can provide temporary or permanent changes in nerve activity. However, traditional chemical treatment modalities such as chemical ablation at the site of the celiac plexus offer modest pain management with a multitude of side effects, as described above.

Embodiments herein are directed to systems and methods that are designed to provide comprehensive pain management while lessening the side effects to a patient. The systems and methods employ use of endoscopic ultrasound-guided radiofrequency ablation and sensing of nerve tissue within the celiac plexus. The location of the celiac plexus near the walls of the stomach and small intestine lends itself well to access through these walls. Delivery of a narrow diameter radiofrequency catheter within an ultrasound endoscope and through the stomach or intestinal wall to the site of the celiac plexus can provide a robust localized system for detecting placement of the radiofrequency catheter at the site of the celiac plexus using ultrasound and for delivery of a radiofrequency energy and for sensing one or more electrical properties of the celiac plexus during the course of a treatment procedure with a radiofrequency energy.

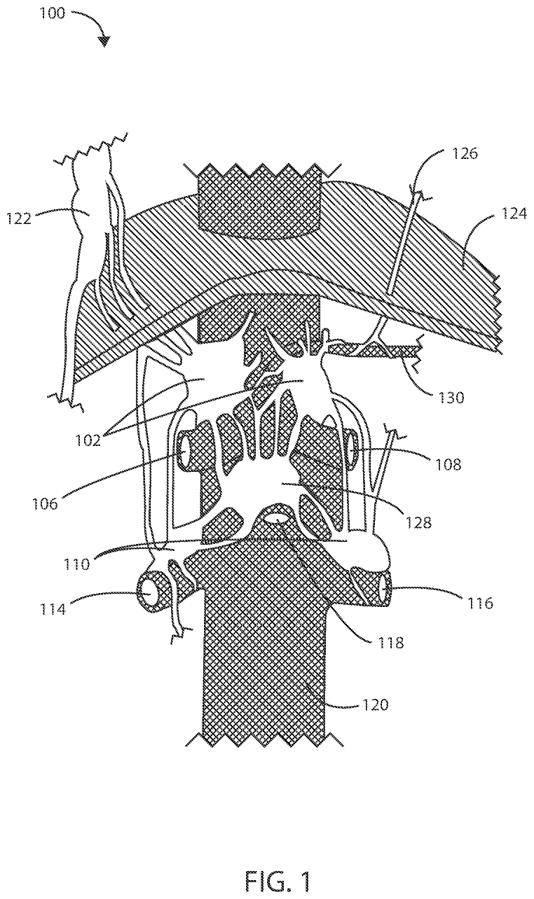

Referring now to FIG. 1, a schematic anterolateral view of a celiac plexus 100 treatment site is shown in accordance with various embodiments herein. The celiac plexus 100 is located in the upper abdomen in an anterior and lateral position with respect to the aorta 120, and can be found disposed anywhere along the aorta 120 at the level of the T12-L2 vertebrae. The celiac plexus 100 is nested within and around various critical arteries, including the common hepatic artery 106, the splenic artery 108, the right renal artery 114, the left renal artery 116, the superior mesenteric artery 118, and the left gastric artery 130. The celiac plexus 100 includes various interconnected ganglia, including but not to be limited to, the left and right celiac ganglia 102, the left and right aortic renal ganglia 110, and the superior mesenteric ganglion 128. The local anatomy around the celiac plexus 100 further includes the splanchnic nerve 122, the vagus nerve 126, and the diaphragm 124.

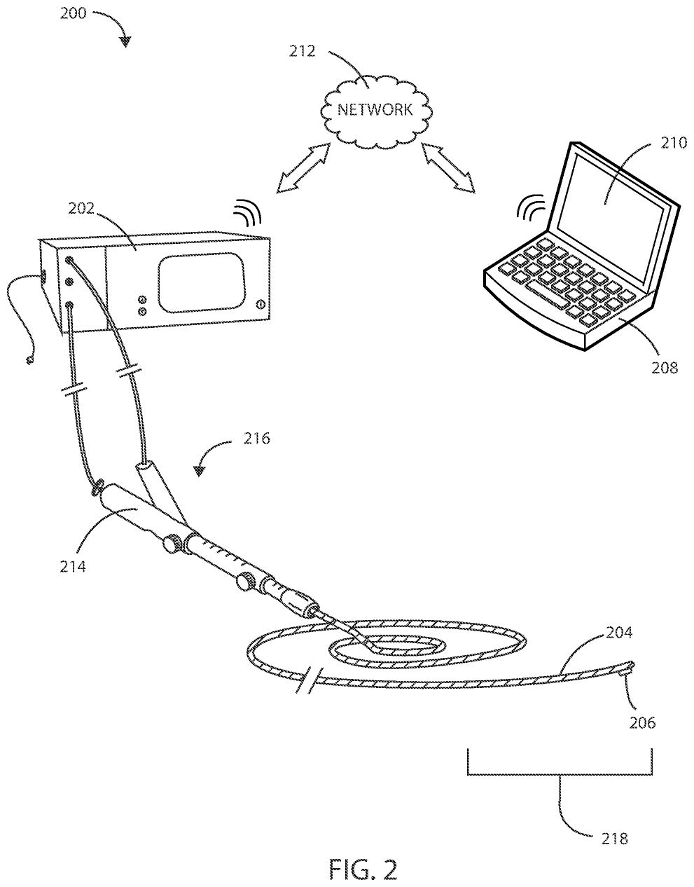

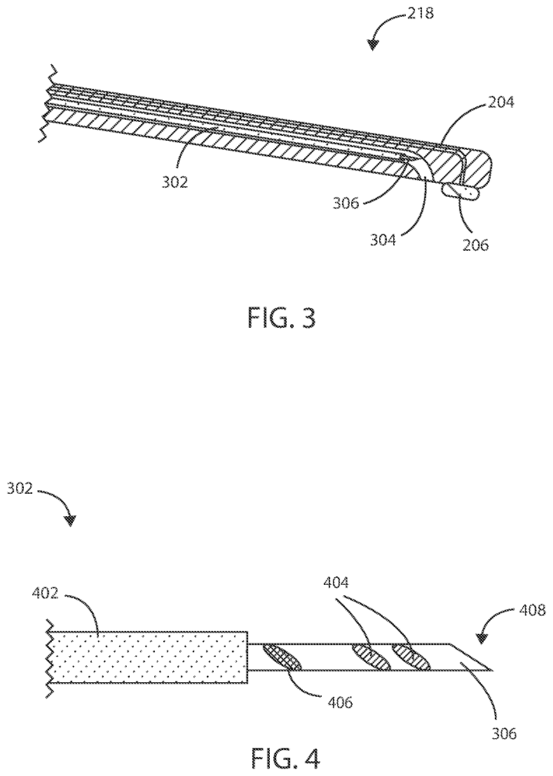

The embodiments herein include ultrasound-guided ablation and sensing systems having components configured for improving pain management for patients with upper abdominal pain using targeted neurolysis of one or more regions of the celiac plexus. Referring now to FIG. 2, a schematic view of the components of a system 200 for endoscopic ultrasound-guided ablation and sensing is shown in accordance with various embodiments herein. The system 200 includes a system controller 202, an ultrasound endoscope 204, and a radiofrequency catheter configured to be disposed within the ultrasound endoscope. The radiofrequency catheter (not shown in FIG. 2) is described further in reference to FIGS. 3-8. The system controller 202 is configured to be in electrical communication with the ultrasound endoscope 204 and the radiofrequency catheter. In various embodiments, the ultrasound endoscope 204 further can include one or more accessories, including a camera, a light source, one or more irrigation lumens, one or more aspiration lumens, one or more drug delivery lumens, and the like.

The ultrasound endoscope 204 includes an ultrasound transducer 206. The ultrasound transducer 206 is configured to be in electrical communication with a power supply. The power supply can be a part of system controller 202 or a separate component in electrical communication with system controller 202. In various embodiments, the ultrasound transducer 206 can be configured to emit ultrasonic sound waves as longitudinal waves in an orientation parallel to the direction of propagation. In other embodiments, the ultrasound transducer 206 can be configured to emit ultrasonic sound waves as transverse waves in an orientation perpendicular to the direction of propagation. The ultrasound endoscope 204 can be configured to localize the vascular structures in and around the vicinity of the celiac plexus using ultrasonic sound waves emitted from the ultrasound transducer 206. The ultrasound endoscope 204 can be configured to provide real-time data regarding the position of the ultrasound endoscope 204 throughout a treatment procedure.

The ultrasound endoscope 204 can include a proximal end 216 and a distal end 218. The proximal end 216 can include a manifold 214. Manifold 214 can include a number of connections and conduits for various electrical components that can be used to place the ultrasound endoscope 204 and radiofrequency catheter in electrical communication with the system controller 202. Manifold 214 can further define one or more lumens configured to receive a radiofrequency catheter, where the radiofrequency catheter can be slidably engaged within the lumen for extension out of and retraction into the ultrasound endoscope 204 at the distal end 218.

The system 200 can further include a computer 208 having a display 210 communicatively coupled to the system controller 202 via a network 212, either via wireless connection or hard-wired connection. In various embodiments, the network 212 can include a wireless network, the Internet, a public or private data network, including packet switched data networks or non-packet switched data networks. System controller 202 can include one or more of a radiofrequency generator, an ultrasound generator, a power supply, and a potentiostat. In various embodiments, a single potentiostat can be utilized for delivery of a current pulse. In other embodiments, at least two potentiostats can be utilized where one can be configured to detect sub-millivolt action potentials of the celiac plexus on a sub-millisecond timescale and the other potentiostat can be utilized for delivery of a current pulse.

The distal end 218 of the ultrasound endoscope can be configured to be inserted into the stomach or small intestine of a patient for localization of the region of the celiac plexus and for advancing a radiofrequency catheter to the nerve tissue of the celiac plexus. Referring now to FIG. 3, the distal end 218 of the ultrasound endoscope 204 is shown in accordance with various embodiments herein. The ultrasound endoscope 204 can define a lumen 304 and can include an ultrasound transducer 206 disposed along the distal end 218. A radiofrequency catheter 302 can be configured to be disposed within the lumen 304 of the ultrasound endoscope 204. The radiofrequency catheter 302 can be slidably engaged within the lumen 304 such that it can be advanced from within the lumen 304 towards the site of the celiac plexus to provide a treatment procedure and can be retracted back into the ultrasound endoscope upon completion of a treatment procedure. The radiofrequency catheter 302 can include an insulative sheath (described further below) disposed about one or more flexible needle catheters 306 configured to be disposed within the lumen 304 defined by the radiofrequency catheter 302. It will be appreciated that each needle catheter 306 can be stowed in a retracted position within the insulative sheath until deployment at or near the site of the celiac plexus.

The radiofrequency catheter 302 can include a variety of configurations in accordance with the embodiments herein. Referring now to FIG. 4, a schematic view of a distal tip of a radiofrequency catheter 302 is shown. The radiofrequency catheter 302 can include an insulative sheath 402 disposed about one or more needle catheters 306 at a distal tip 408 of the radiofrequency catheter 302. In the embodiments herein, the needle catheters 306 can include those having a tapered end, a pointed end, a conical end, and the like. The needle catheters 306 can be configured to penetrate through the stomach wall or intestinal wall and toward a site of the celiac plexus 100. Insulative sheath 402 can be made from one or more insulative and flexible materials, including, but not to be limited to, one or more polymers or copolymers such as polytetrafluoroethylene, polyurethane, polyethylenetheraphtalate, silicone, polyhexamethylene, polyvinylchloride, and derivatives thereof.

The radiofrequency catheter 302 can include one or more electrode pairs disposed on the needle catheters 306 of the radiofrequency catheter 302. The one or more electrode pairs can be configured to be placed at or near the site of the celiac plexus. In various embodiments, the one or more electrode pairs can be placed in direct contact with the nerve tissue of the celiac plexus. In other embodiments, the one or more electrode pairs can be placed in the vicinity of the nerve tissue of the celiac plexus. The radiofrequency catheter 302 shown in FIG. 4 includes a first electrode pair 404 disposed along the needle catheter 306 of radiofrequency catheter 302 at the distal tip 408. In various embodiments, the radiofrequency catheter 302 further can include at least one of a second electrode pair, a third electrode pair, or a fourth electrode pair. It will be appreciated that the radiofrequency catheters described herein can include from one, two, three, four, five, six, seven, eight, nine, or ten electrode pairs. In various embodiments, the radiofrequency catheter 302 can include more than ten electrode pairs.

The electrode pairs disposed on the needle catheters of the radiofrequency catheters can be adjacent to one another and can be electrically insulated from one another. In various embodiments, the electrode pairs herein can include at least one of circular band pairs, interdigitated ring pairs, concentric ring-disk pairs, or parallel pairs. In various embodiments, the electrode pairs can be made of one or more materials such as, but not to be limited to, silver, gold, platinum, iridium, glassy carbon, or iridium oxide-coated stainless steel, or derivatives or alloys thereof.

The one or more electrode pairs disposed on the radiofrequency catheters described herein are configured to both deliver a radiofrequency energy to the nerve tissue of the celiac plexus and to sense one or more electrical properties of the celiac plexus throughout a treatment procedure. By way of example, the system 200 is configured to sense a baseline electrical property of the celiac plexus to verify placement of the first electrode pair at the celiac plexus. Upon confirmation that the radiofrequency catheter is in position at the celiac plexus, the system 200 is further configured to deliver a radiofrequency (RF) energy to the celiac plexus through the first electrode pair following a predetermined treatment procedure. The electrode pairs can be used to sense an electrical property of the celiac plexus either periodically during the course of delivering a radiofrequency energy to the celiac plexus, or at the termination of a treatment procedure. Thus, the system 200 is configured to sense a during-treatment electrical property at predetermined time intervals to monitor treatment progression and/or a post-treatment electrical property of the celiac plexus to verify neurolysis of the celiac plexus, as will be described further below.

In various embodiments, it will be appreciated that the placement of the radiofrequency catheter at the celiac plexus may require more than one attempt at proper localization. If it is determined that the radiofrequency catheter is not at the site of the celiac plexus, then the radiofrequency catheter may be further advanced, retracted, or altogether removed and repositioned at the site of the celiac plexus. In various embodiments, one or more electrical properties can be monitored in real time during placement to confirm localization at the celiac plexus. Values for the one or more electrical properties of the celiac plexus can be compared against known values for the electrical property within a given subject or within a given population of subjects. In various embodiments, a plurality of electrical properties can be tested to confirm placement of the radiofrequency catheter at the site of the celiac plexus.

Various electrical properties can be sensed by the electrode pairs herein, including one of a baseline electrical property, a during-treatment electrical property, or a post-treatment electrical property. The sensed baseline electrical property can include a value for an electrical property at the time preceding a treatment procedure. The sensed baseline electrical property can be sensed and recorded at a time immediately prior to initiating a treatment procedure or within a predetermined time frame prior to initiating a treatment procedure. The sensed during-treatment electrical property can include a value for an electrical property at any time interval between the initiation of a treatment procedure through termination of a treatment procedure. The sensed post-treatment electrical property can include a value for an electrical property upon termination of a treatment procedure. In some embodiments, the post-treatment electrical property can be obtained immediately upon termination of a treatment procedure or within a predetermined time frame following termination of a treatment procedure.

In various embodiments, the baseline electrical property, the during-treatment electrical property, or the post-treatment electrical property can include an electrical potential, an action potential, an impedance, permittivity, conductivity, or the like. It will be appreciated that in some embodiments herein, the baseline electrical property, the during-treatment electrical property, and the post-treatment electrical property sensed by the one or more electrode pairs are the same. In other embodiments, the baseline electrical property, the during-treatment electrical property, and the post-treatment electrical property sensed by the one or more electrode pairs are different, such that they are independently one of either an action potential or an impedance, or any combination thereof.

In various embodiments, the radiofrequency catheters herein can include a reference electrode 406 placed proximal to the electrode pairs for monitoring and reducing noise in the system. It will be appreciated that in various embodiments, radiofrequency catheter 302 can include more than one reference electrode, such as two, three, four, five, or more reference electrodes. The reference electrodes suitable for use herein can include those made from silver or other highly conductive materials. It will be appreciated that in some embodiments the radiofrequency catheters herein can be disposable.

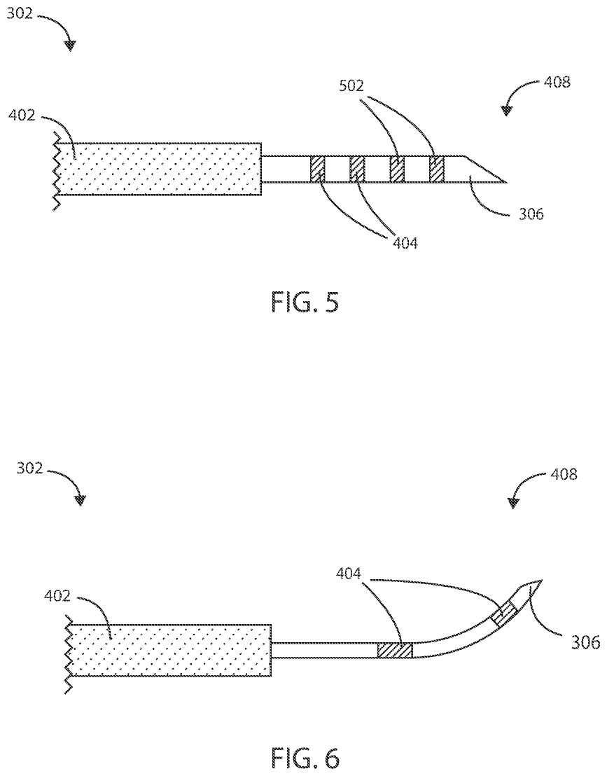

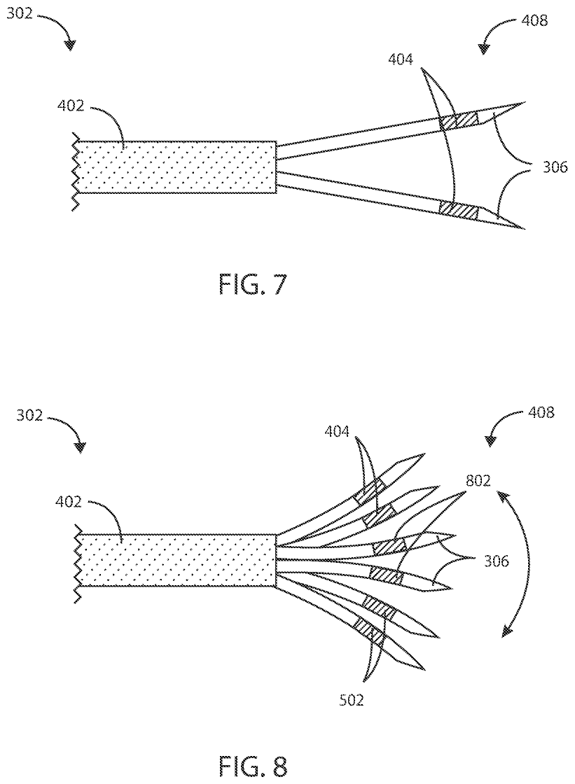

Additional configurations for the radiofrequency catheters suitable for use herein can include those shown in FIGS. 5-8. In the configuration shown in FIG. 5, radiofrequency catheter 302 can include a first electrode pair 404 and a second electrode pair 502 disposed at the distal tip 408. The first electrode pair 404 and a second electrode pair 502 shown in FIG. 5 can include circular band pairs that span the entire circumference of the needle catheter 306, or that span a portion of the circumference of the needle catheter 306. In the configuration shown in FIG. 6, radiofrequency catheter 302 can include a first electrode pair 404 disposed at the distal tip 408, where the distal tip 408 is configured to be curvilinear relative the longitudinal axis of the radiofrequency catheter 302. In the configuration shown in FIG. 7, radiofrequency catheter 302 can include a first electrode pair 404, where each individual electrode in the pair is disposed on a separate needle catheter 306 at the distal tip 408. In the configuration shown in FIG. 8, radiofrequency catheter 302 can include multiple needles catheters 306, each including at least one electrode disposed at the distal tip 408. The radiofrequency catheter 302 of FIG. 8 includes a first electrode pair 404, a second electrode pair 502, and a third electrode pair 802 where each individual electrode is each disposed on a separate needle catheter 306 at the distal tip 408. In the radiofrequency catheter 302 shown in FIG. 8, the individual needle catheters 306 can be configured to be curvilinear relative the longitudinal axis of the radiofrequency catheter 302.

The radiofrequency catheters and electrodes herein can be configured to be in electrical communication with a radiofrequency generator. The radiofrequency generator can be a part of system controller 202 or a separate component in electrical communication with system controller 202. In various embodiments, the radiofrequency catheters and electrodes can be further configured to be in electrical communication with a potentiostat. The potentiostat can be a part of system controller 202 or a separate component in electrical communication with system controller 202. The radiofrequency catheters and electrodes herein further can be configured to be in electrical communication with a power supply for supplying a voltage or current to the electrode pairs disposed on the radiofrequency catheter. The power supply can be a part of system controller 202 or a separate component in electrical communication with system controller 202.

The radiofrequency catheters and electrode pairs herein can be configured to deliver an alternating current pulse through one or more electrode pairs to the site of the celiac plexus for targeted radiofrequency ablation of the nerve tissue. In various embodiments, the current pulse delivered to the site of the celiac plexus can include a 2 milliamp (mA) pulse. In various embodiments, the current pulse delivered to the site of the celiac plexus can include a current pulse that can be greater than or equal to 0.1 mA, 0.2 mA, 0.3 mA, 0.4 mA, 0.5 mA, 0.6 mA, 0.7 mA, 0.8 mA, 0.9 mA, 1 mA, 2 mA, 3 mA, 4 mA, 5 mA, 6 mA, 7 mA, 8 mA, 9 mA, 10 mA, 11 mA, 12 mA, 13 mA, 14 mA, 15 mA, 16 mA, 17 mA, 18 mA, 19 mA, or 20 mA, or can be an amount falling within a range between any of the foregoing.

The radiofrequency catheters and electrode pairs herein can be configured to deliver a voltage pulse through one or more electrode pairs to the site of the celiac plexus for targeted radiofrequency ablation of the nerve tissue. In various embodiments, the voltage pulse delivered to the site of the celiac plexus can include a 10 mV pulse. In various embodiments, the current pulse delivered to the site of the celiac plexus can include a voltage pulse that can be greater than or equal to 1 mV, 2 mV, 3 mV, 4 mV, 5 mV, 6 mV, 7 mV, 8 mV, 9 mV, 10 mV, 11 mV, 12 mV, 13 mV, 14 mV, 15 mV, 16 mV, 17 mV, 18 mV, 19 mV, or 20 mV, or can be an amount falling within a range between any of the foregoing.

The radiofrequency catheters and electrode pairs herein can be configured to deliver a current pulse or a voltage pulse at a range of frequencies, including from 100 kilohertz (kHz) to 3 megahertz (MHz; i.e., 3000 kHz) to the site of the celiac plexus. In some embodiments, the radiofrequency catheters and electrode pairs herein can be configured to deliver a current pulse or a voltage pulse at a range of frequencies, including from 250 kHz to 750 kHz to the site of the celiac plexus. In various embodiments, delivering a radiofrequency energy to the celiac plexus can include delivery of a current pulse or a voltage pulse at a frequency of greater than or equal to 100 kHz, 200 kHz, 300 kHz, 400 kHz, 500 kHz, 600 kHz, 700 kHz, 800 kHz, 900 kHz, 1000 kHz, 1100 kHz, 1200 kHz, 1300 kHz, 1400 kHz, 1500 kHz, 1600 kHz, 1700 kHz, 1800 kHz, 1900 kHz, 2000 kHz, 2100 kHz, 2200 kHz, 2300 kHz, 2400 kHz, 2500 kHz, 2600 kHz, 2700 kHz, 2800 kHz, 2900 kHz, or 3000 kHz, or can be an amount falling within a range between any of the foregoing.

The configuration of the radiofrequency catheter and/or needle catheters disposed therein can include those with a narrow diameter to keep the puncture site at the stomach or intestinal wall small and the side effects of delivering a catheter to the site of the celiac plexus to a minimum. In various embodiments, the radiofrequency catheters and needles herein can include those with an outer diameter of from 0.1 millimeters (mm) to 1.5 mm. In various embodiments, the radiofrequency catheters and needles herein can include those with an outer diameter of from 0.50 millimeters (mm) to 1.0 mm. In some embodiments, the diameter of the radiofrequency catheters and needles can be greater than or equal to 0.05 mm, 0.1 mm, 0.15 mm, 0.2 mm, 0.25 mm, 0.3 mm, 0.35 mm, 0.4 mm, 0.45 mm, 0.5 mm, 0.55 mm, 0.6 mm, 0.65 mm, 0.7 mm, 0.75 mm, 0.8 mm, 0.85 mm, 0.9 mm, 0.95 mm, 1.0 mm, 1.05 mm, 1.1 mm, 1.15 mm, 1.2 mm, 1.25 mm, 1.3 mm, 1.35 mm, 1.4 mm, 1.45 mm, or 1.50 mm, or can be an amount falling within a range between any of the foregoing.

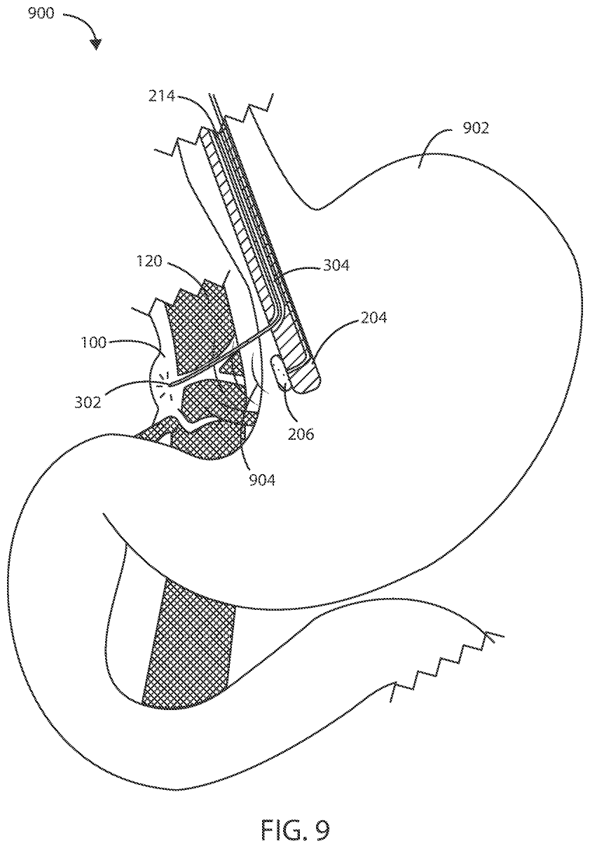

As discussed herein, an ultrasound-guided endoscope and radiofrequency catheter in accordance with the embodiments herein can be inserted into the stomach or small intestine via the esophagus and placed in proximity to the site of the celiac plexus. Referring now to FIG. 9 a schematic view of the components of a system at or near a location of a treatment site is shown in accordance with various embodiments herein. The ultrasound endoscope 204 is configured to be inserted into the stomach 902 in proximity to the celiac plexus 100 and near the aorta 120. The ultrasound endoscope is configured to emit ultrasonic sound waves 904 from the ultrasound transducer 206 in a direction toward the celiac plexus 100 to identify vascular structures and anatomy indicative of the site of the celiac plexus 100. The radiofrequency catheter 302 is configured to be disposed within a lumen 304 of the ultrasound endoscope 204. The radiofrequency catheter 302 is further configured to be advanced from within the lumen 304 through a wall of the stomach or small intestine and towards the site of the celiac plexus 100 to provide a treatment procedure. The treatment procedure can include delivery of at least one of a current pulse or a voltage pulse across one or more frequency ranges as described elsewhere herein, to ablate the nerve tissue of the celiac plexus.

### Methods

Many different methods are contemplated herein, including, but not limited to, methods of making, methods of using, and the like. Aspects of system/device operation described elsewhere herein can be performed as operations of one or more methods in accordance with various embodiments herein.

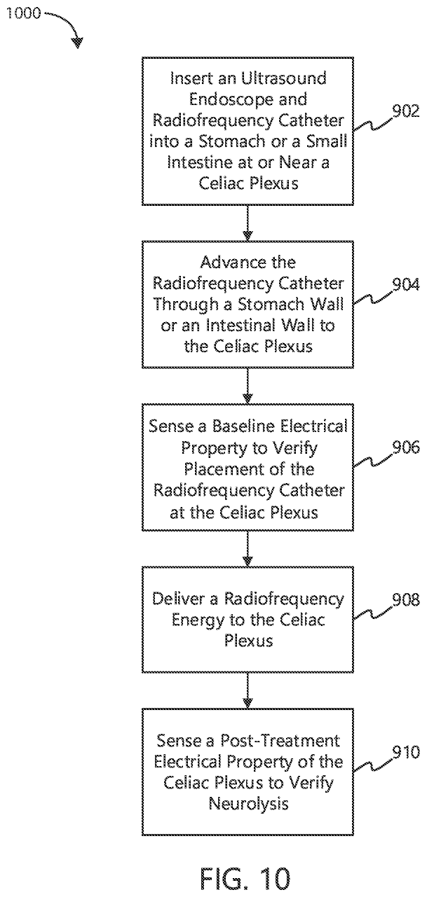

Referring now to FIG. 10, a flow diagram of a method is shown in accordance with various embodiments herein. In the embodiment shown in FIG. 10, a method 1000 for endoscopic ultrasound-guided ablation and sensing in a subject is included. The method 1000 includes inserting an ultrasound endoscope and radiofrequency catheter into a stomach or a small intestine of the subject at or near a site of a celiac plexus at 902. The ultrasound endoscope can include an ultrasound transducer as described herein. The radiofrequency catheter is configured to be disposed within the ultrasound endoscope, where the radiofrequency catheter can include a first electrode pair disposed on the radiofrequency catheter. The first electrode pair can be configured to be placed at or near the site of the celiac plexus. In some embodiments the first electrode pair can be placed in direct contact with the nerve tissue of the celiac plexus. In other embodiments, the first electrode pair can be place in the vicinity of the nerve tissue of the celiac plexus. The method 1000 can include advancing the radiofrequency catheter through a stomach wall or an intestinal wall to the site of the celiac plexus at 904. The method 1000 can include sensing a baseline electrical property to verify placement of the first electrode pair at the celiac plexus at 906. The method can include delivering a radiofrequency (RF) energy to the celiac plexus through the first electrode pair at 908 and sensing a post-treatment electrical property of the celiac plexus to verify neurolysis of the celiac plexus at 910.

In various embodiments, it will be appreciated that the placement of the radiofrequency catheter at or near the site of the celiac plexus using the methods herein may require more than one attempt at proper localization. If it is determined that the radiofrequency catheter is not at the site of the celiac plexus, then the radiofrequency catheter may be further advanced, retracted, or altogether removed and repositioned at the site of the celiac plexus. Advancement or retraction can be of different distances, but in some cases can be at least about 0.5, 1, 2, 3, 4, 5, 8, 10, 15, 20, 30 or 40 mm or more, or a distance falling within a range between any of the foregoing. It will be appreciated that multiple attempts may be made in order to achieve proper placement. In various embodiments, one or more electrical properties can be monitored in real time during placement at or near the site of the celiac plexus to confirm localization. Values for the one or more electrical properties of the celiac plexus can be compared against known values for the electrical property within a given subject or within a given population of subjects. In various embodiments, a plurality of electrical properties can be tested to confirm placement of the radiofrequency catheter at the site of the celiac plexus.

In various embodiments, delivering the radiofrequency energy to the celiac plexus can include delivery of the radiofrequency energy until an electrical property of the celiac plexus reaches a predetermined value substantially indicative of complete neurolysis. In some embodiments, delivery of the radiofrequency energy can include delivery of the radiofrequency energy until an electrical property of the celiac plexus reaches a value that is at least 10% different than the baseline value for the electrical property. In some embodiments, delivery of the radiofrequency energy can include delivery of the radiofrequency energy until an electrical property of the celiac plexus reaches a value that is at least 10%, 15%, 20%, 25%, 30%, 35%, 40%, 45%, 50%, 60%, 70%, 80%, 90%, 100%, 125%, 150%, 175%, 200%, 300%, 400%, 500%, or 1000%, or more different than the baseline value for the electrical property, or can be an amount falling within a range between any of the foregoing. In various embodiments, the value for the electrical property of the celiac plexus can increase or decrease relative to the baseline value for the electrical property.

The method further can include sensing one or more during-treatment electrical properties at various predetermined time intervals during the course of a treatment procedure. By way of example, the initial delivery of a radiofrequency energy can include applying a current pulse or a voltage pulse to the celiac plexus for from 1 second (sec) to 30 sec and then temporarily pausing the delivery of the radiofrequency energy to sense a during-treatment electrical property before proceeding with the delivery of the radiofrequency energy to the celiac plexus. Sensing a during-treatment electrical property can be performed one or more times during the course of a treatment procedure. In various embodiments, the delivery of a radiofrequency energy to the celiac plexus can be temporarily paused after applying a current pulse or a voltage pulse to the celiac plexus for from 1 sec, 2 sec, 3 sec, 4 sec, 5 sec, 6 sec, 7 sec, 8 sec, 9 sec, 10 sec, 11 sec, 12 sec, 13 sec, 14 sec, 15 sec, 16 sec, 17 sec, 18 sec, 19 sec, 20 sec, 21 sec, 22 sec, 23 sec, 24 sec, 25 sec, 26 sec, 27 sec, 28 sec, 29 sec, 30 sec, 31 sec, 32 sec, 33 sec, 34 sec, 35 sec, 36 sec, 37 sec, 38 sec, 39 sec, 40 sec, 41 sec, 42 sec, 43 sec, 44 sec, 45 sec, 46 sec, 47 sec, 48 sec, 49 sec, 50 sec, 51 sec, 52 sec, 53 sec, 54 sec, 55 sec, 56 sec, 57 sec, 58 sec, 59 sec, or 60 sec, or can be an amount falling within a range between any of the foregoing.

The various electrical properties that can be sensed by the electrode pairs herein include one of a baseline electrical property, a during-treatment electrical property, or a post-treatment electrical property. In various embodiments, the baseline electrical property, the during-treatment electrical property, or the post-treatment electrical property can each include either an action potential or an impedance. It will be appreciated that in some embodiments herein, the baseline electrical property, the during-treatment electrical property, and the post-treatment electrical property sensed by the one or more electrode pairs are all the same, such as all being an action potential or all being an impedance. In other embodiments, the baseline electrical property, the during-treatment electrical property, and the post-treatment electrical property sensed by the one or more electrode pairs are different, such that they are independently one of either an action potential or an impedance, or any combination thereof.

The methods herein can include delivering a radiofrequency energy to the site of the celiac plexus using the radiofrequency catheters and electrode pairs as described. Delivering a radiofrequency energy can include delivering an alternating current pulse through one or more electrode pairs to the site of the celiac plexus for targeted radiofrequency ablation of the nerve tissue. In various embodiments, the current pulse delivered to the site of the celiac plexus can include a 2 milliamp pulse. In various embodiments, delivering a radiofrequency energy to the celiac plexus can include delivering a current pulse that can be greater than or equal to 0.1 mA, 0.2 mA, 0.3 mA, 0.4 mA, 0.5 mA, 0.6 mA, 0.7 mA, 0.8 mA, 0.9 mA, 1 mA, 2 mA, 3 mA, 4 mA, 5 mA, 6 mA, 7 mA, 8 mA, 9 mA, 10 mA, 11 mA, 12 mA, 13 mA, 14 mA, 15 mA, 16 mA, 17 mA, 18 mA, 19 mA, or 20 mA, or can be an amount falling within a range between any of the foregoing.

The methods can include using the radiofrequency catheters and electrode pairs described herein to deliver a voltage pulse through one or more electrode pairs to the site of the celiac plexus for targeted radiofrequency ablation of the nerve tissue. In various embodiments, the voltage pulse delivered to the site of the celiac plexus can include a 10 millivolt (mV) pulse. In various embodiments, delivering a radiofrequency energy to the celiac plexus can include delivering a voltage pulse that can be greater than or equal to 1 mV, 2 mV, 3 mV, 4 mV, 5 mV, 6 mV, 7 mV, 8 mV, 9 mV, 10 mV, 11 mV, 12 mV, 13 mV, 14 mV, 15 mV, 16 mV, 17 mV, 18 mV, 19 mV, or 20 mV, or can be an amount falling within a range between any of the foregoing.

The methods herein can include using radiofrequency catheters and electrode pairs described herein to deliver a current pulse or a voltage pulse at a range of frequencies, including from 100 kilohertz (kHz) to 3 megahertz (MHz; i.e., 3000 kHz) to the site of the celiac plexus. In some embodiments, the radiofrequency catheters and electrode pairs herein can be configured to deliver a current pulse or a voltage pulse at a range of frequencies, including from 250 kHz to 750 kHz to the site of the celiac plexus. In various embodiments, delivering a radiofrequency energy to the celiac plexus can include delivery of a current pulse or a voltage pulse at a frequency of greater than or equal to 100 kHz, 200 kHz, 300 kHz, 400 kHz, 500 kHz, 600 kHz, 700 kHz, 800 kHz, 900 kHz, 1000 kHz, 1100 kHz, 1200 kHz, 1300 kHz, 1400 kHz, 1500 kHz, 1600 kHz, 1700 kHz, 1800 kHz, 1900 kHz, 2000 kHz, 2100 kHz, 2200 kHz, 2300 kHz, 2400 kHz, 2500 kHz, 2600 kHz, 2700 kHz, 2800 kHz, 2900 kHz, or 3000 kHz, or can be an amount falling within a range between any of the foregoing.

### Sensing an Action Potential of the Celiac Plexus

The electrode pairs described herein can be configured with the dual ability to deliver a radiofrequency energy to perform ablation of nerve tissue within the celiac plexus and to sense one or more electrical properties of the celiac plexus. The one or more electrical properties of the celiac plexus can be sensed before, during, or after a treatment procedure and can be stored to memory for future analysis. As described elsewhere herein, the electrical properties can include an action potential or an impedance of the nerve tissue within the celiac plexus.

Without wishing to be bound by any particular theory, it is believed that a potential can exist across the cell membrane of most cell types within the body. A resting potential exists within most cells at rest, where the potential difference across the membrane typically exists such that the interior of the cell is negative with respect to the exterior of the cell. Approximate values for the resting potential of cell types found in and near the celiac plexus includes neurons (−70 mV), skeletal muscle cells (−80 mV), cardiac muscle cells (−90 mV), smooth muscle cells (−60 mV), and adipose cells (−40 mV). An action potential is initiated as a wave or impulse along various locations along the cell membrane and results in a rapid rise of fall in membrane polarity with respect to the resting potential. A membrane potential can change in response to a chemical, electrical, or physical stimulus. An electrical stimulus can include an applied voltage pulse or an applied current pulse to the site of a nerve tissue such as that found in the celiac plexus.

Action potential sensing can be used to determine the tissue type in the region surrounding the radiofrequency catheter in order to confirm placement at or within the nerve tissue of the celiac plexus. Upon initial placement of the radiofrequency catheter at the site of the celiac plexus, an action potential can be sensed to provide a clinician with an indication that the radiofrequency catheter is directly contacting or in close vicinity to the celiac plexus. The sensed action potential can be compared against a known value or set of values indicative of an action potential or resting potential characteristic of the celiac plexus and the surrounding tissues. The sensed action potential can be combined with a sensed impedance (described further below), and can be utilized by the physician or clinician in real-time to monitor progression of neurolysis, confirm continued placement at the proper location of the celiac plexus, and confirm partial or complete ablation of the celiac plexus.

Sensing an action potential can include passively sensing a potential of the nerve tissue, such as sensing an a baseline action potential or a resting potential prior to a treatment procedure. Alternatively, or in combination, sensing an action potential can include sensing an evoked action potential at a given time during a treatment procedure or at any time following termination of a treatment procedure. The evoked action potential can result following the application of a current pulse or a voltage pulse to the nerve tissue of the celiac plexus. Over the course of a treatment procedure, the sensed action potential can be monitored and used as a guide to determine the effectiveness and extent of neurolysis of the celiac plexus.

### Electrochemical Impedance Spectroscopy

Electrochemical impedance spectroscopy (EIS) can be used to determine the tissue type in the region surrounding the radiofrequency catheter in order to confirm placement at or within the nerve tissue of the celiac plexus and to monitor the progression of a treatment procedure. EIS can be used in the methods herein for sensing an impedance of the celiac plexus to provide a clinician with an indication that the radiofrequency catheter is directly contacting or in close vicinity to the celiac plexus. Without wishing to be bound by any particular theory, it is believed that EIS is a useful tool for differentiating various tissue types.

Impedance can be calculated according to Ohm's law, which provides that electrical potential (V), current (I), and impedance (Z) are interrelated (V=IR or V=IZ, where R is resistance in an idea resistor). Thus, by knowing one variable (e.g., such as a supplied current) and measuring another (e.g., such as measuring voltage drop), the impedance (Z) can be calculated. In some embodiments herein, impedance can be measured by measuring the voltage and dividing by the applied current. Within the body, impedance can be influenced by a number of factors, including but not limited to components in contact with the electrode pairs, such as muscle tissue, fat, connective tissue, nerve tissue, and bone; cell density, cell size; local electrolyte concentrations, etc.

It will be appreciated that different tissues will have different impedances at a given frequency and as such, in some embodiments, measuring impedance at one or more frequencies at any given location is contemplated to differentiate tissue type. Upon placement of the radiofrequency catheter at the site of the celiac plexus, a voltage can be delivered to the region and an impedance can be determined at one or more frequencies. The measured impedance can be compared against a known value or set of values indicative of an impedance characteristic of the celiac plexus and the surrounding tissues. In some embodiments, the delivered current or voltage can be a constant current or a constant voltage. In other embodiments, the delivered current or voltage can be a pulsed current or a pulsed voltage.

The systems herein can be configured to sense an impedance of the celiac plexus by delivering an alternating current of from 1 to 10 (milliamp) mAmp through a range of frequencies of from 1 hertz (Hz) to 1000 Hz to the site of the celiac plexus. In various embodiments, the alternating current can be delivered to the site of the celiac plexus including a current that that can be greater than or equal to 0.1 mA, 0.2 mA, 0.3 mA, 0.4 mA, 0.5 mA, 0.6 mA, 0.7 mA, 0.8 mA, 0.9 mA, 1 mA, 2 mA, 3 mA, 4 mA, 5 mA, 6 mA, 7 mA, 8 mA, 9 mA, 10 mA, 11 mA, 12 mA, 13 mA, 14 mA, 15 mA, 16 mA, 17 mA, 18 mA, 19 mA, or 20 mA, or can be an amount falling within a range between any of the foregoing. The range of frequencies suitable for use in determining an impedance of the celiac plexus can include from 1 Hz, 2 Hz, 3 Hz, 4 Hz, 5 Hz, 6 Hz, 7 Hz, 8 Hz, 9 Hz, 10 Hz, 20 Hz, 30 Hz, 40 Hz, 50 Hz, 75 Hz, 100 Hz, 125 Hz, 150 Hz, 175 Hz, 200 Hz, 225 Hz, 250 Hz, 275 Hz, 300 Hz, 325 Hz, 350 Hz, 375 Hz, 400 Hz, 425 Hz, 450 Hz, 475 Hz, 500 Hz, 525 Hz, 550 Hz, 575 Hz, 600 Hz, 625 Hz, 650 Hz, 675 Hz, 700 Hz, 725 Hz, 750 Hz, 775 Hz, 800 Hz, 825 Hz, 850 Hz, 875 Hz, 900 Hz, 925 Hz, 950 Hz, 975 Hz, or 1000 Hz, or can be an amount falling within a range between any of the foregoing.

### Treatment Procedures

Many different treatment procedures are contemplated herein, for use with the systems and methods described. The various treatment procedures can be utilized in one or more treatments to accomplish neurolysis of the celiac plexus. The treatment procedures include at least one of chronoamperometry, chronopotentiometry, and/or electrical impedance spectroscopy (EIS) (as described above). In various embodiments, the treatment procedures utilized in the treatments herein can include alternating chronoamperometry with EIS. In other embodiments, the treatment procedures utilized in the treatments herein can include alternating chronopotentiometry with EIS. Alternating chronoamperometry and chronopotentiometry independently with EIS can enhance the ability of the clinician to identify correct placement of the electrodes and to detect nerve signals before, during, and at the completion of a treatment procedure.

The duration of the treatment procedures described herein can include applying a treatment to the celiac plexus over the course of from 1 second to 120 seconds. In some embodiments, the treatment procedure can be applied to the celiac plexus for from 1 sec, 2 sec, 3 sec, 4 sec, 5 sec, 6 sec, 7 sec, 8 sec, 9 sec, 10 sec, 11 sec, 12 sec, 13 sec, 14 sec, 15 sec, 16 sec, 17 sec, 18 sec, 19 sec, 20 sec, 21 sec, 22 sec, 23 sec, 24 sec, 25 sec, 26 sec, 27 sec, 28 sec, 29 sec, 30 sec, 31 sec, 32 sec, 33 sec, 34 sec, 35 sec, 36 sec, 37 sec, 38 sec, 39 sec, 40 sec, 41 sec, 42 sec, 43 sec, 44 sec, 45 sec, 46 sec, 47 sec, 48 sec, 49 sec, 50 sec, 51 sec, 52 sec, 53 sec, 54 sec, 55 sec, 56 sec, 57 sec, 58 sec, 59 sec, 60 sec, 61 sec, 62 sec, 63 sec, 64 sec, 65 sec, 66 sec, 67 sec, 68 sec, 69 sec, 70 sec, 71 sec, 72 sec, 73 sec, 74 sec, 75 sec, 76 sec, 77 sec, 78 sec, 79 sec, 80 sec, 81 sec, 82 sec, 83 sec, 84 sec, 85 sec, 86 sec, 87 sec, 88 sec, 89 sec, 90 sec, 91 sec, 92 sec, 93 sec, 94 sec, 95 sec, 96 sec, 97 sec, 98 sec, 99 sec, 100 sec, 101 sec, 102 sec, 103 sec, 104 sec, 105 sec, 106 sec, 107 sec, 108 sec, 109 sec, 110 sec, 111 sec, 112 sec, 113 sec, 114 sec, 115 sec, 116 sec, 117 sec, 118 sec, 119 sec, or 120 sec, or can be an amount falling within a range between any of the foregoing. In various embodiments, the treatment procedures herein can be applied to the celiac plexus for greater than 120 sec.

### Chronoamperometry

During a chronoamperometry procedure, electrical energy is applied at a particular voltage (electrical potential) between at least a first pair of electrodes at the site of the celiac plexus and a current response can be monitored (variation in current over time) by the same first pair of electrodes that supplied the electrical energy. One or more cycles of electrical stimulation can be applied during a course of a treatment to achieve partial or complete neurolysis of the celiac plexus. A chronoamperometry procedure can involve stepping through various voltage potentials from a low voltage potential to a high voltage potential during a course of a treatment procedure. It will be appreciated that multiple electrode pairs can be utilized during a chronoamperometry procedure to strengthen the signal-to-noise ratio. In some embodiments, the chronoamperometry procedures herein can include the repeated application of two different voltage potentials in an alternating manner for a defined number of cycles during the course of a treatment procedure. Various voltages suitable for use in the treatment procedures herein are described in more detail above.

### Chronopotentiometry

During a chronopotentiometry procedure, electrical energy is applied at a particular current strength (amperage) between at least a first pair of electrodes at the site of the celiac plexus and an electrical potential response can be monitored (variation in voltage over time) by the same first pair of electrodes that supplied the electrical energy. One or more currents of different magnitude can be applied during a course of a treatment to achieve partial or complete neurolysis of the celiac plexus. A chronoamperometry procedure can involve stepping through various current strengths from a low current to a high current during a course of a treatment. It will be appreciated that multiple electrode pairs can be utilized during a chronopotentiometry procedure to strengthen the signal-to-noise ratio. In some embodiments, the chronoamperometry procedures herein can include the repeated application of two different currents magnitudes in an alternating manner for a defined number of cycles during the course of a treatment procedure. Various current magnitudes suitable for use in the treatment procedures herein are described in more detail above.

### System Components

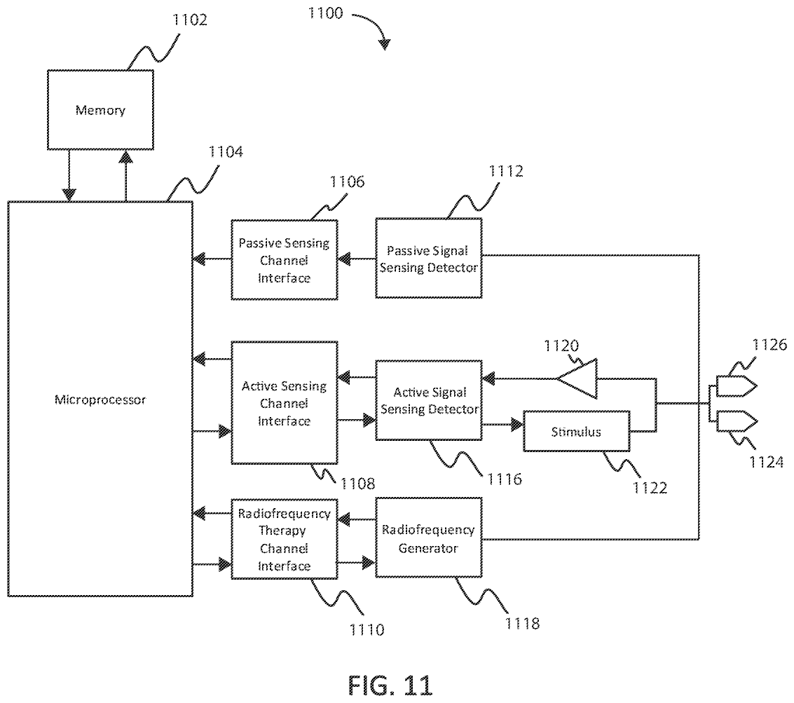

Various components can be provided in the embodiments of the system for endoscopic ultrasound-guided celiac plexus ablation and sensing described herein. Referring now to FIG. 11, a schematic view of elements of a system for endoscopic ultrasound-guided ablation and sensing is shown in accordance with some embodiments herein. It will be appreciated that some embodiments can include additional elements beyond those shown in FIG. 11. In addition, some embodiments may lack some elements shown in FIG. 11. The radiofrequency catheter disposed within the ultrasound endoscope can deliver a radiofrequency energy through one or more radiofrequency channels and can sense an electrical property of the celiac plexus through one or more sensing channels. A microprocessor 1104 communicates with a memory 1102 via a bidirectional data bus. The memory 1102 can include read only memory (ROM) and/or random access memory (RAM) for program storage and RAM for data storage.

The system for endoscopic ultrasound-guided celiac plexus ablation and sensing can include sensing and radiofrequency ablation channels for at least a first pair of electrodes 1124 and 1126. The system can include a passive sensing channel interface 1106 configured to be in electrical communication with a passive signal sensing detector 1112 and in unidirectional communication with a port of microprocessor 1104. The system can include an active sensing channel interface 1108 configured to be in electrical communication with an active signal sensing detector 1116 and can communicate bidirectionally with a port of microprocessor 1104. The active signal sensing channel can include a sensing amplifier 1120, an output circuit to provide a stimulus 1122, where the output circuit provides a stimulus to electrodes 1124 and 1126 and the active signal sensing detector 1116 is in electrical communication with the electrodes 1124 and 1126. The system can also include a radiofrequency therapy channel interface 1110 configured to be in electrical communication with a radiofrequency therapy generator 1118 and can provide a radiofrequency energy to the electrodes 1124 and 1126. The radiofrequency therapy channel interface 1110 can be configured to communicate bidirectionally with a port of microprocessor 1104.

Aspects may be better understood with reference to the following examples. These examples are intended to be representative of specific embodiments, but are not intended as limiting the overall scope of embodiments herein.

### EXAMPLES

### Example 1: Experimental Conditions

A canine subject was prepared for ablation of the celiac plexus under general anesthesia. An ultrasound endoscope including an ultrasound transducer and a radiofrequency catheter configured to be disposed within the ultrasound endoscope was inserted into through the mouth of the canine subject and advanced through the esophagus and into the stomach. The celiac plexus was located using ultrasound guidance. Once the celiac plexus was located, the radiofrequency catheter was advanced out of the lumen of the ultrasound endoscope and through the stomach wall to the site of the celiac plexus. The radiofrequency catheter included a circular band electrode pair made from a platinum-iridium (Pt—Ir) metal alloy that was placed in direct contact with the celiac plexus.

A chronoamperometry ablation treatment procedure was performed for 90 minutes on the subject following an initial sensing of a baseline action potential in the celiac plexus. Four independent ablations were performed, where each ablation procedure lasted for 2 minutes followed by from 10 minutes to 20 minutes of rest.

### Example 2: Chronoamperometry Ablation Treatment Procedure

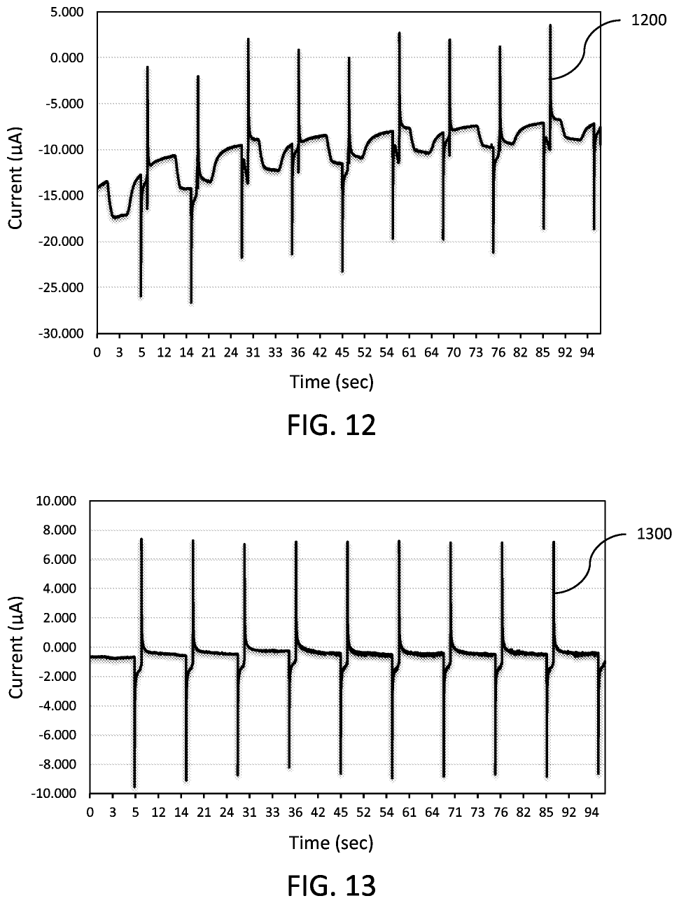

Contact between the electrode pair and celiac plexus was confirmed by passively sensing a baseline action potential prior to initiating the ablation treatment procedure. The baseline action potential of the celiac plexus is shown as plot 1200 in FIG. 12. The baseline action potential was passively sensed for 2 minutes, and a current was reported from within a range of −30.00 microamps (μA) to 5 μA. A first ablation was initiated following sensing of the baseline action potential. A 10 millivolt pulse was applied to the site of the celiac plexus for 2 minutes at a frequency of 100 Hz. The action potential of the celiac plexus was passively sensed for the duration of the first ablation and plotted as a function of sensed current in microamps (μA) versus time in seconds (sec). The action potential of the celiac plexus during the first ablation of the treatment procedure is shown as plot 1300 in FIG. 13. The action potential of the celiac plexus during the first ablation procedure was reported from within a range of −10.00 μA to 8 μA.

A second, third, and fourth ablation was performed on the celiac plexus following the completion of the first ablation. A 10 millivolt pulse was applied to the site of the celiac plexus for 2 minutes at a frequency of 100 Hz for each of the second, third, and fourth ablations. A 10-20 minute rest period was included between each successive ablation. The action potential of the celiac plexus was passively sensed for the duration of the second, third, and fourth ablations and plotted as a function of sensed current in microamps (μA) versus time in seconds (sec).

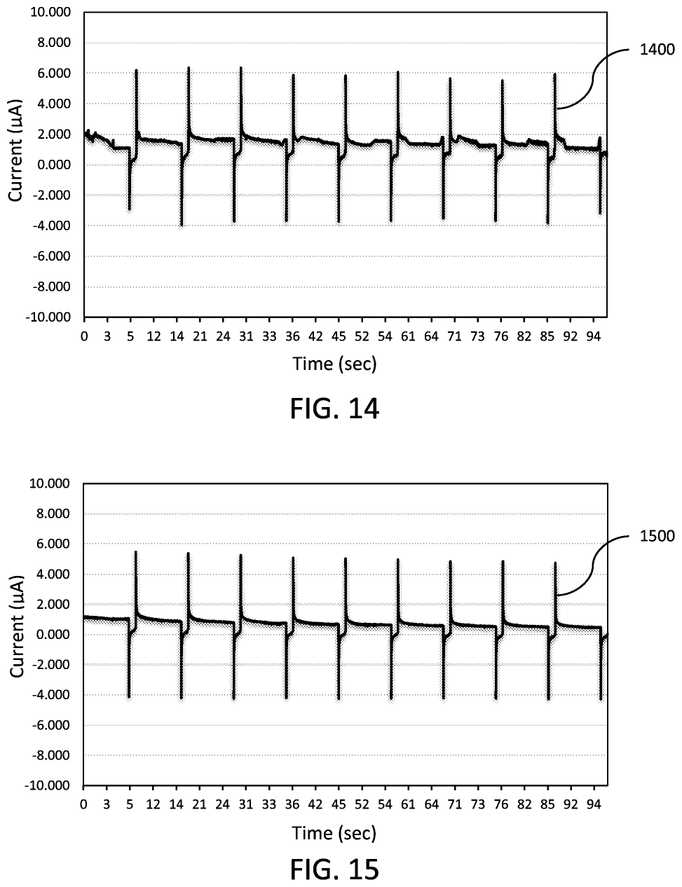

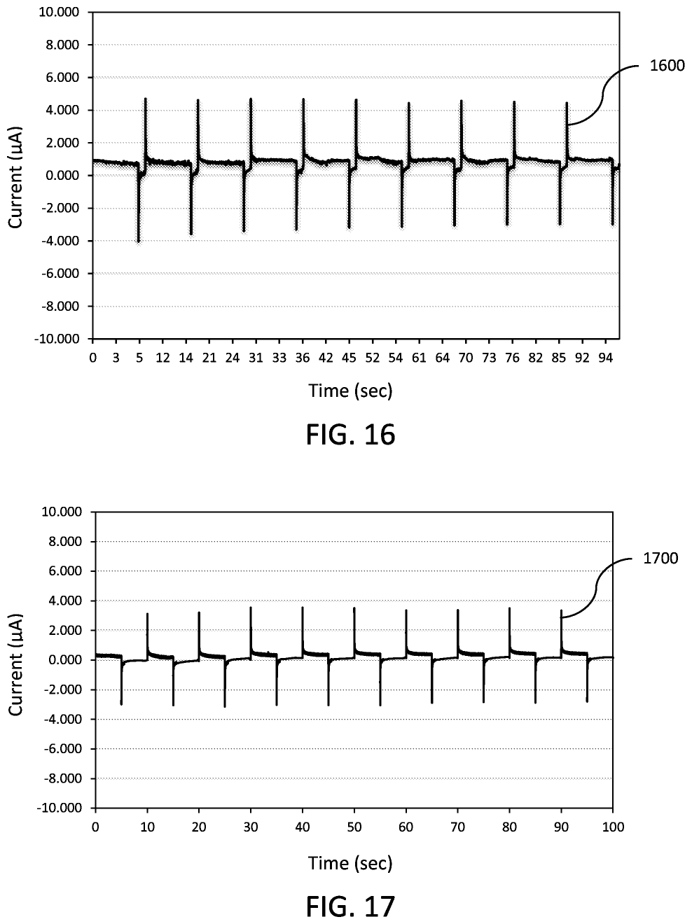

The action potential of the celiac plexus during the second ablation of the treatment procedure is shown as plot 1400 in FIG. 14. The action potential of the celiac plexus during the second ablation procedure was reported from within a range of −6.00 μA to 8 μA. The action potential of the celiac plexus during the third ablation of the treatment procedure is shown as plot 1500 in FIG. 15. The action potential of the celiac plexus during the third ablation procedure was reported from within a range of −6.00 μA to 6 μA. The action potential of the celiac plexus during the second ablation of the treatment procedure is shown as plot 1600 in FIG. 16. The action potential of the celiac plexus during the third ablation procedure was reported from within a range of −5.00 μA to 5 μA.

### Example 3: Chronoamperometry Controls

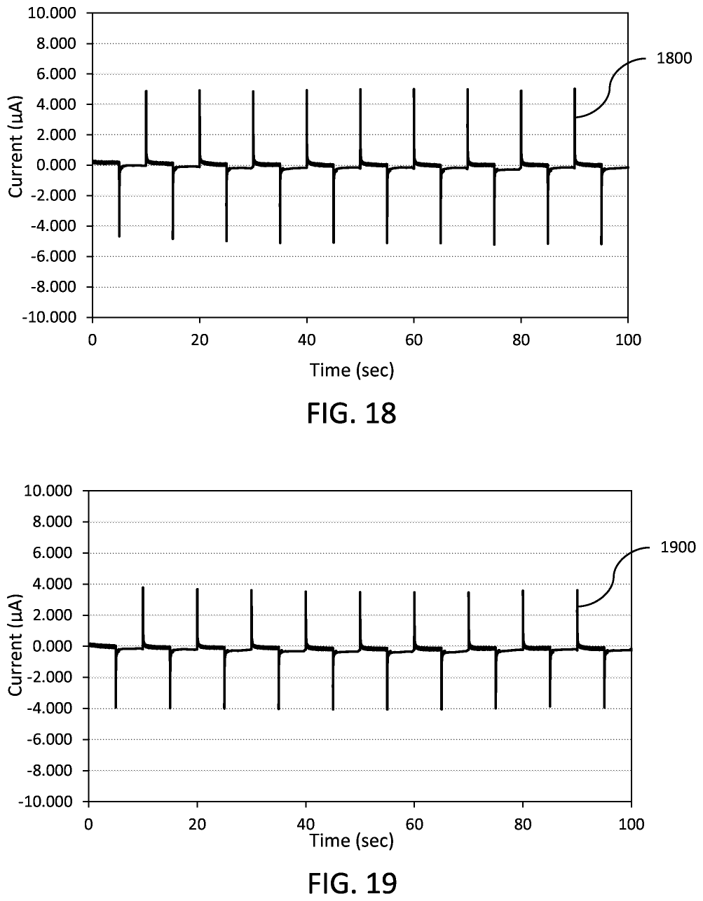

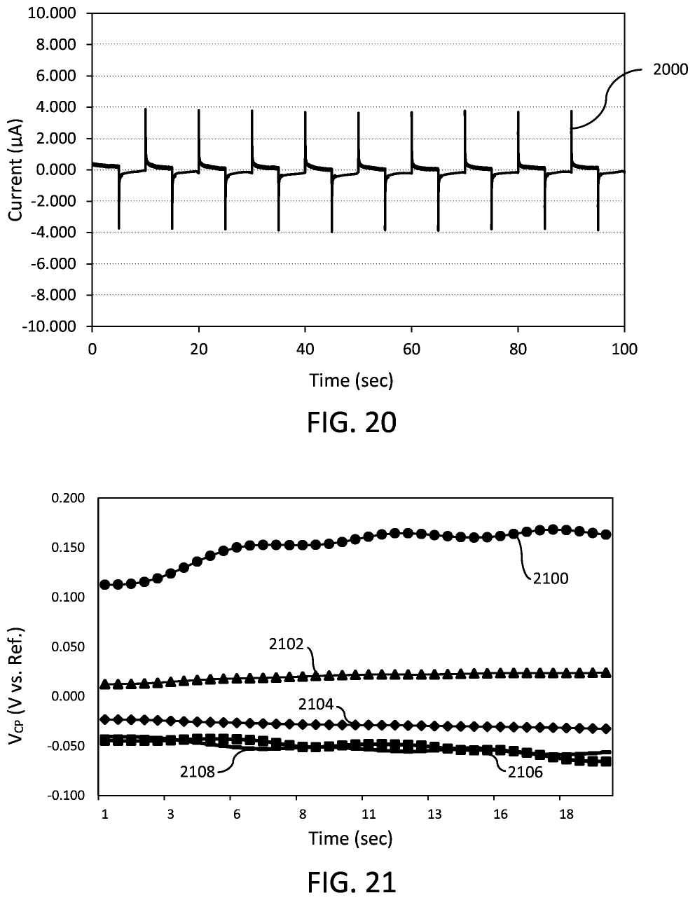

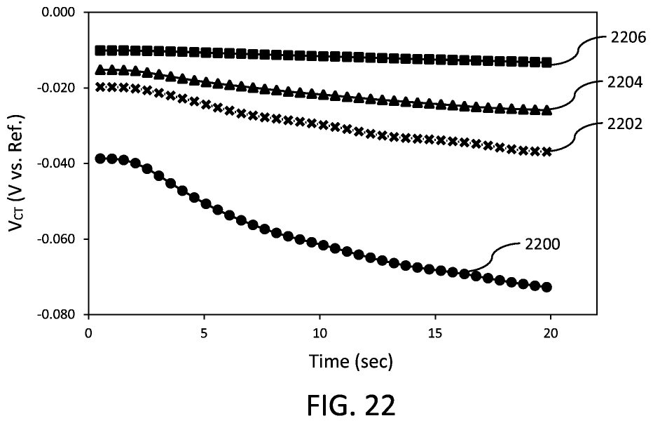

Chronoamperometry controls were performed on smooth muscle tissue, liver tissue, skeletal muscle tissue, and adipose tissue. Each tissue type was located using ultrasound guidance. Contact between the electrode pair and each of the control tissue types was confirmed by passively sensing a baseline action potential. The baseline action potential of smooth muscle tissue is shown as plot 1700 in FIG. 17. The baseline action potential for smooth muscle tissue was passively sensed for 2 minutes, and a current was reported from within a range of −4.00 microamps (μA) to 4 μA. The baseline action potential of liver tissue is shown as plot 1800 in FIG. 18. The baseline action potential was for liver tissue passively sensed for 2 minutes, and a current was reported from within a range of −6.00 μA to 6 μA. The baseline action potential of skeletal muscle tissue is shown as plot 1900 in FIG. 19. The baseline action potential was for skeletal tissue passively sensed for 2 minutes, and a current was reported from within a range of −4.00 μA to 4 μA. The baseline action potential of adipose tissue is shown as plot 2000 in FIG. 20. The baseline action potential was for adipose tissue passively sensed for 2 minutes, and a current was reported from within a range of −4.00 μA to 4 μA.

### Example 4: Open Circuit Potential of the Celiac Plexus During Chronoamperometry Ablation

The open circuit potential of the celiac plexus was determined by sensing the voltage of the celiac plexus versus a reference potential during the chronoamperometry ablation treatment procedure. Without wishing to be bound by any particular theory, it is believed that the open circuit potential measurements can be used to determine the presence and extent of tissue ablation, such as neurolysis of a nervous tissue like the celiac plexus. A plot of the open circuit potential of the celiac plexus V_{CP} (V. vs. Ref) as a function of time (sec.) during the chronoamperometry ablation treatment procedure is shown in FIG. 21.

Plot 2100 (solid circles) shows the open circuit potential of the celiac plexus before any ablative current had been applied. Plot 2102 (solid triangles) shows the open circuit potential of the celiac plexus after the first 2 minute ablation treatment had been applied to the celiac plexus. Plot 2104 (solid diamond) shows the open circuit potential of the celiac plexus after the second 2 minute ablation treatment had been applied to the celiac plexus. Plot 2106 (solid squares) shows the open circuit potential of the celiac plexus after the third 2 minute ablation treatment had been applied to the celiac plexus. Plot 2108 (solid dashes) shows the open circuit potential of the celiac plexus after the fourth 2 minute ablation treatment had been applied to the celiac plexus. The decrease in the open circuit potential during the course of the chronoamperometry ablation treatment procedure after the first, second, third, and fourth ablations indicate that the celiac plexus tissue underwent significant neurolysis during the treatment procedure.

### Example 5: Open Circuit Potential of the Control Tissues During Chronoamperometry Ablation

The open circuit potentials of various control tissues, including smooth muscle tissue, liver tissue, skeletal muscle tissue, and adipose tissue, were determined by sensing the voltage of the control tissues versus a reference potential. A plot of the open circuit potential of the control tissues V_{CT} (V. vs. Ref.) as a function of time (sec.) is shown in FIG. 21.

Plot 2200 (solid circles) shows the open circuit potential of smooth muscle tissue. Plot 2202 (solid x symbol) shows the open circuit potential of adipose tissue. Plot 2204 (solid triangle) shows the open circuit potential of liver tissue. Plot 2206 (solid squares) shows the open circuit potential of skeletal muscle.

### Example 6: Electrical Impedance of the Celiac Plexus During Chronoamperometry Ablation

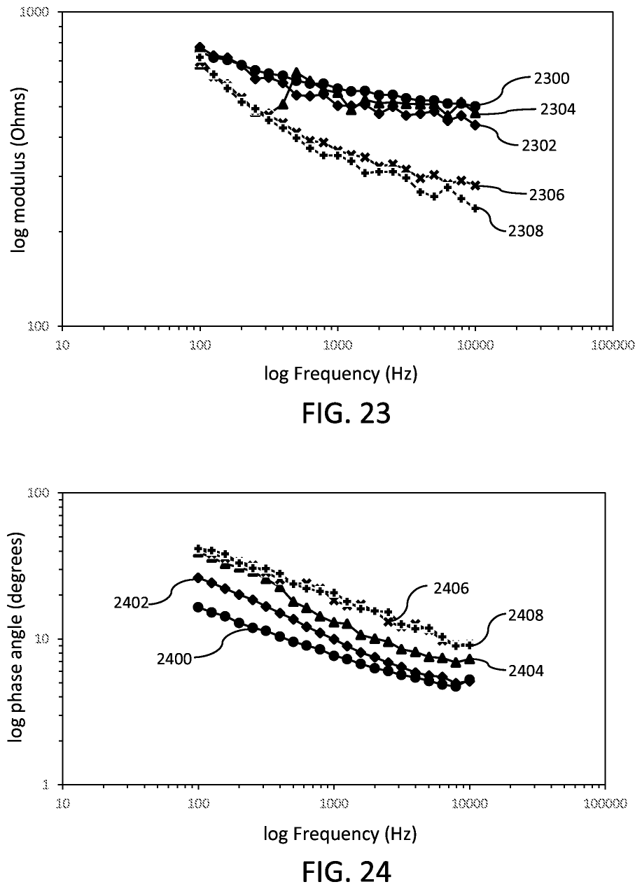

Electrical impedance spectroscopy of the celiac plexus was sensed during the duration of the chronoamperometry ablation procedure. A pair of Pt—Ir electrodes was inserted at the site of the celiac plexus and a 10 millivolt (mV) rms voltage was applied before the ablation treatment procedure and after each subsequent 2-minute ablation period. Following each ablation period, the electrical impedance values were obtained over a frequency range of from 100 Hz to 10000 Hz. Both the magnitude (plotted as the log of the modulus of electrical impedance in Ohms) and phase angle (plotted as the log of degrees) of the impedance signals were plotted against the log of frequency (Hz), as shown in FIG. 23 and FIG. 24, respectively.

FIG. 23 shows a graph of the log of the modulus of electrical impedance in Ohms versus the log of frequency in Hz. Plot 2300 (solid circles) shows the magnitude of the electrical impedance as a function of frequency for the celiac plexus before any ablative current had been applied. Plot 2302 (solid diamonds) shows the magnitude of the electrical impedance as a function of frequency for the celiac plexus after the first 2 minute ablation treatment had been applied to the celiac plexus. Plot 2304 (solid triangles) shows the magnitude of the electrical impedance as a function of frequency for the celiac plexus after the second 2 minute ablation treatment had been applied to the celiac plexus. Plot 2306 (solid x symbols) shows the magnitude of the electrical impedance as a function of frequency for the celiac plexus after the third 2 minute ablation treatment had been applied to the celiac plexus. Plot 2308 (solid plus symbols) shows the magnitude of the electrical impedance as a function of frequency for the celiac plexus after the fourth 2 minute ablation treatment had been applied to the celiac plexus.

FIG. 24 shows a graph of the log of the phase angle of electrical impedance in degrees versus the log of frequency in Hz. Plot 2400 (solid circles) shows the phase angle of the electrical impedance as a function of frequency for the celiac plexus before any ablative current had been applied. Plot 2402 (solid diamonds) shows the phase angle of the electrical impedance as a function of frequency for the celiac plexus after the first 2 minute ablation treatment had been applied to the celiac plexus. Plot 2404 (solid triangles) shows the phase angle of the electrical impedance as a function of frequency for the celiac plexus after the second 2 minute ablation treatment had been applied to the celiac plexus. Plot 2406 (solid x symbols) shows the phase angle of the electrical impedance as a function of frequency for the celiac plexus after the third 2 minute ablation treatment had been applied to the celiac plexus. Plot 2408 (solid plus symbols) shows the phase angle of the electrical impedance as a function of frequency for the celiac plexus after the fourth 2 minute ablation treatment had been applied to the celiac plexus.

Thus, the decrease in impedance and phase angle during the course of treatment confirms that the nerve tissue of the celiac plexus was changed. The changes include both physical and chemical changes in the celiac plexus and are detected as a reduction in the barrier for voltage passing between the electrodes.

### Example 7: Electrical Impedance of the Control Tissues During Chronoamperometry Ablation

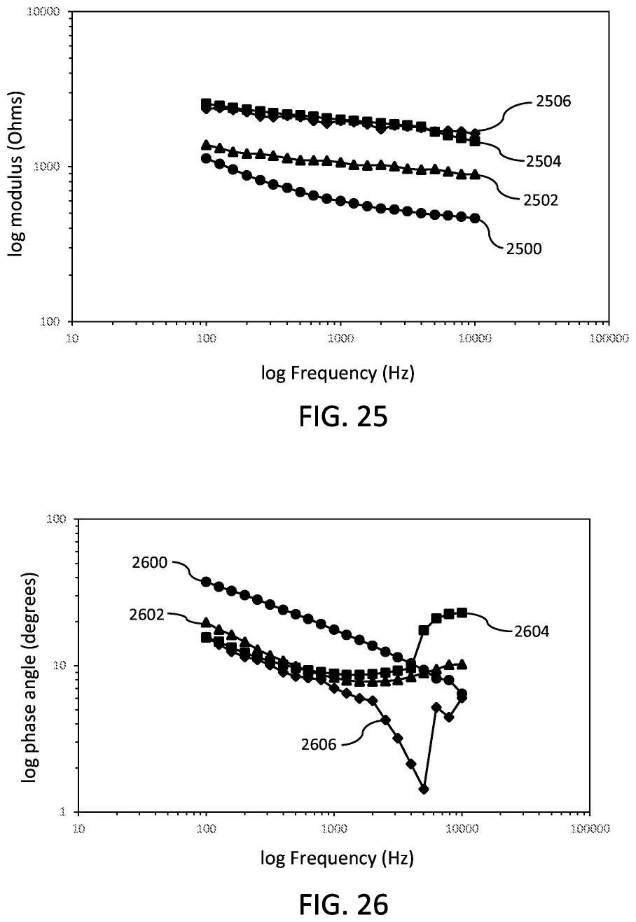

Electrical impedance spectroscopy of the control tissues, including smooth muscle tissue, liver tissue, skeletal muscle tissue, and adipose tissue, were sensed and recorded. A 10 millivolt (mV) rms voltage was applied to the site of each control tissue and the electrical impedance was monitored as a function of frequency within a range of frequencies from 100 Hz to 10000 Hz. Both the magnitude (plotted as the log of the modulus of electrical impedance in Ohms) and phase angle (plotted in degrees) of the impedance signals were plotted against the log of frequency (Hz), as shown in FIG. 25 and FIG. 26, respectively.

FIG. 25 shows a graph of the log of the modulus of electrical impedance in Ohms versus the log of frequency in Hz. Plot 2500 (solid circles) shows the magnitude of the electrical impedance as a function of frequency for the smooth muscle tissue. Plot 2502 (solid triangles) shows the magnitude of the electrical impedance as a function of frequency for the liver tissue. Plot 2504 (solid squares) shows the magnitude of the electrical impedance as a function of frequency for the skeletal muscle tissue. Plot 2506 (solid diamond) shows the magnitude of the electrical impedance as a function of frequency for the adipose tissue.

FIG. 26 shows a graph of the log of the phase angle of electrical impedance in degrees versus the log of frequency in Hz. Plot 2600 (solid circles) shows the phase angle of the electrical impedance as a function of frequency for the smooth muscle tissue. Plot 2602 (solid triangles) shows the phase angle of the electrical impedance as a function of frequency for the liver tissue. Plot 2604 (solid squares) shows the phase angle of the electrical impedance as a function of frequency for the skeletal muscle. Plot 2606 (solid diamonds) shows the phase angle of the electrical impedance as a function of frequency for the adipose tissue.

It should be noted that, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

All publications and patent applications in this specification are indicative of the level of ordinary skill in the art to which this invention pertains. All publications and patent applications are herein incorporated by reference to the same extent as if each individual publication or patent application was specifically and individually indicated by reference.

As used herein, the recitation of numerical ranges by endpoints shall include all numbers subsumed within that range (e.g., 2 to 8 includes 2.1, 2.8, 5.3, 7, etc.).

The headings used herein are provided for consistency with suggestions under 37 CFR 1.77 or otherwise to provide organizational cues. These headings shall not be viewed to limit or characterize the invention(s) set out in any claims that may issue from this disclosure. As an example, although the headings refer to a “Field,” such claims should not be limited by the language chosen under this heading to describe the so-called technical field. Further, a description of a technology in the “Background” is not an admission that technology is prior art to any invention(s) in this disclosure. Neither is the “Summary” to be considered as a characterization of the invention(s) set forth in issued claims.

The embodiments described herein are not intended to be exhaustive or to limit the invention to the precise forms disclosed in the following detailed description. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

1. A system for endoscopic ultrasound-guided ablation and sensing comprising:
an ultrasound endoscope comprising an ultrasound transducer;
a radiofrequency catheter configured to be disposed within the ultrasound endoscope, the radiofrequency catheter comprising
a first electrode pair disposed on the radiofrequency catheter, the first electrode pair configured to be placed at or near at a site of a celiac plexus;
wherein the system is configured to
sense a baseline electrical property to verify placement of the first electrode pair at the celiac plexus;
deliver a radiofrequency energy to the celiac plexus through the first electrode pair; and
sense a post-treatment electrical property of the celiac plexus to verify neurolysis of the celiac plexus.

2. The system of claim 1, further comprising a system controller in electrical communication with the ultrasound endoscope and the radiofrequency catheter.

3. The system of claim 1, wherein the baseline electrical property is at least one of an action potential and an impedance.

4. The system of claim 1, wherein the post-treatment electrical property is at least one of an action potential and an impedance.

5. The system of claim 1, wherein delivering a radiofrequency energy to the celiac plexus comprises the first electrode pair being configured to deliver an alternating electric current pulse or a voltage pulse having a frequency of from 250 kilohertz (kHz) to 3 megahertz (MHz) to the site of the celiac plexus.

6. The system of claim 1, wherein delivering a radiofrequency energy to the celiac plexus comprises the first electrode pair being configured to deliver an alternating electric current pulse or a voltage pulse having a frequency of from 250 kHz to 750 kHz to the site of the celiac plexus.

7. The system of claim 1, wherein the radiofrequency catheter comprises a needle catheter at a distal tip of the radiofrequency catheter.

8. The system of claim 1, wherein the radiofrequency catheter comprises more than one needle catheters at a distal tip of the radiofrequency catheter.

9. The system of claim 1, wherein the radiofrequency catheter further comprises at least one of a second electrode pair, a third electrode pair, or a fourth electrode pair.

10. The system of claim 1, wherein the first electrode pair comprises at least one of circular band pairs, interdigitated ring pairs, concentric ring-disk pairs, or parallel pairs.

11. The system of claim 1, wherein the first electrode pair is made of one or more materials comprising silver, gold, platinum, iridium, glassy carbon, or iridium oxide-coated stainless steel, or derivatives or alloys thereof.

12. The system of claim 1, the radiofrequency catheter further comprising at least one reference electrode.

13. A method for endoscopic ultrasound-guided ablation and sensing in a subject comprising:
inserting an ultrasound endoscope and a radiofrequency catheter into a stomach or a small intestine of the subject at or near a site of a celiac plexus;
wherein the ultrasound endoscope comprises an ultrasound transducer, and wherein the radiofrequency catheter is configured to be disposed within the ultrasound endoscope, the radiofrequency catheter comprising
a first electrode pair disposed on the radiofrequency catheter, the first electrode pair configured to be placed at or near the site of the celiac plexus;
advancing the radiofrequency catheter through a stomach wall or an intestinal wall to the site of the celiac plexus;
sensing a baseline electrical property to verify placement of the first electrode pair at the celiac plexus;
delivering a radiofrequency energy to the celiac plexus through the first electrode pair; and
sensing a post-treatment electrical property of the celiac plexus to verify neurolysis of the celiac plexus.

14. The method of claim 13, further comprising delivering the radiofrequency energy to the celiac plexus until an electrical property of the celiac plexus reaches a predetermined value indicative of neurolysis.

15. The method of claim 13, wherein the baseline electrical property is at least one of an action potential and an impedance.

16. The method of claim 13, wherein the post-treatment electrical property is at least one of an action potential and an impedance.

17. The method of claim 13, further comprising delivering the radiofrequency energy to the celiac plexus until a post-treatment action potential signal of the celiac plexus reaches a value of less than 10% of a baseline action potential of the celiac plexus.

18. The method of claim 13, wherein delivering a radiofrequency energy to the celiac plexus comprises delivering alternating electric current pulse or a voltage pulse having a frequency of from 250 kilohertz (kHz) to 3 megahertz (MHz) to the site of the celiac plexus.

19. The method of claim 13, further comprising sensing an impedance of the celiac plexus by delivering an alternating current of from 1 to 10 (milliamp) mAmp through a range of frequencies of from 1 hertz (Hz) to 1000 Hz to the site of the celiac plexus.

20. The method of claim 13, further comprising sensing an impedance of the celiac plexus using an electrochemical impedance spectroscopy.
